(19) United States
(12) Patent Application Publication
(10) Pub. No.: US 2015/0126877 A1
(43) Pub. Date: May 7, 2015

(54) Seat Having Integrated EKG-Electrodes

(71) Applicant: Almanaa Trading EST. K.S.A., Riyadh (SA)

(72) Inventors: Peter Roehr, Braunschweig (DE); Joachim Vetter, Salzgitter (DE); Martin Oehler, Braunschweig (DE)

(21) Appl. No.: 14/402,118

(22) PCT Filed: May 16, 2013
(86) PCT No.: PCT/EP2013/001456

§ 371 Date: Nov. 19, 2014

(30) Foreign Application Priority Data
May 19, 2012 (DE) 10 2012 009 973.8

(51) Int. Cl. A61B 5/0408; A61B 5/12; A61B 5/04; A61B 5/22; A61B 5/00; A61B 5/0205; A61B 5/145

(52) U.S. Cl. CPC A61B 5/04085; A61B 5/0205; A61B 5/121; A61B 5/14542; A61B 5/222; A61B 5/742; A61B 5/6891; A61B 5/04012; A61B 5/021
USPC 600/484; 600/509; 600/513; 600/483

(57) ABSTRACT

A bio-measurement data recording device has a seat which has a back surface made of a foam material, a seat surface, and a plurality of back surface sensors embedded in the back surface such that a plurality of leads for an electrocardiogram of a the person can be received. At least two seat surface sensors are embedded in the seat surface. A left hand grip is arranged such that it can be gripped by the person with the left hand and which has a left hand grip sensor or conducting a channel for the electrocardiogram, and a right hand grip is arranged such that it can be gripped by said person with the right hand and which has a right hand grip sensor for conducting a channel for the electrocardiogram. An evaluation unit is connected to the sensors and is configured to ascertain the electrocardiogram.

The invention relates to a bio-measurement data recording device having a seat, an ergometer unit and an evaluation unit.

The bio-measurement data recording device refers especially to an ECG, which is designed to interact with a number of bio-measurement data recording devices such that, in addition to an electrocardiogram (ECG), a variety of bio-measurement data of a person can be recorded without the person having to change seats.

A bio-measurement data recording device according to the preamble is known from DE 10 2010 025 038 A1 as part of a so-called diagnostic garden. The disadvantage of this bio-measurement data recording device is that is not possible to obtain reliable statements concerning the health of the test person's heart.

WO 98/25520 describes a safety device for vehicle drivers, wherein the vehicle control elements are fitted with electrodes. Two of these electrodes are dynamically selected and the driver's heartbeat is recorded from the potential difference of the two electrodes. This is then used to ascertain the driver's emotions. This type of system enables only qualitative statements concerning the change that occurs in the driver.

DE 10 2009 050755 A1 describes how electrodes can be built into a car for the determination of an ECG, so that the driver's pulse can be recorded during the journey. The disadvantage of this type of system is that the quality of the data that can be obtained is not sufficient for making diagnostically reliable statements. As a general rule, it is accepted that the driver is sufficiently healthy to continue driving the vehicle. It also describes how to insert electrodes into the steering wheel: however, this leads to the same problems, as diagnostically reliable data, such as ECG data, cannot be derived from only two measurement data.

Bio-measurement data recording devices in the form of ergometers are a standard component in gyms, many households and in some medical applications, such as electrocardiogram (ECG) exercise test. To these ends, a huge variety of ergometer systems are used.

The disadvantage of known bio-measurement data recording devices is that their use is not very popular. This means that medically useful examinations are conducted either too late or not at all.

The invention aims to reduce the disadvantages of the prior art.

The invention solves the problems by means of a bio-measurement data recording device with the features described in claim 1.

This type of bio-measurement data recording device is designed to record an electrocardiogram while the person concerned sits on the seat and holds one of the two hand grips in each hand. This has the advantage that the person barely notices that an electrocardiogram is being recorded. The inhibition threshold with regards to the use of such equipment is thereby reduced, as it is perceived as more of fun gadget, rather than a medical examination device.

The bio-measurement data recording device, which may also be referred to as a prevention ergometer, may also be configured so that sensors automatically record the vital statistics of the user when they start using the device and the device automatically calculates an individual ergometer programme. The user can then only choose between a sports-related, fitness, weight-loss or medical use. The sequence of the prevention ergometer programme is designed to be interesting, exciting and adventurous for the user so that it leads to increased motivation, but is relaxing at the same time. For the recording of their essential bio-measurement data during their workout, the person in the machine can be integrated into the projected image of a film that plays on the integrated hemispherical screen.

The bio-measurement data recording device is advantageous in that it is easy to use and entertaining: this can influence a change in attitude. In this way, the willingness to use it for precautionary examinations may increase.

In addition to this, the modular structure is also an advantage. The evaluation unit in particular is configured to communicate with a variety of bio-measurement data recording devices. As a result, the bio-measurement data recording device can be extended and adjusted.

Within the scope of the present description, the term foam material should be understood especially to mean a foam material which adapts to the person's back. In particular, the foam material refers to viscoelastic foam material. The seat is preferably inclined backwards at a positive angle from the vertical, such that the person's back lies constantly on the back surface.

The term electrocardiogram should be understood particularly to mean an electrocardiogram with at least four channels, especially with at least five channels. An electrocardiogram can be defined as the recording of voltages which occur due to the curves of stimulation of the human heart. In principle, an individual voltage-time-curve may also be understood to be an electrocardiogram. However, this is not what is meant with regards to the present application. The therapeutic value of electrocardiograms with less than three channels is generally too low.

The back surface sensors are embedded in the foam material in such a way that they enable at least five standard leads of an electrocardiogram. In order for the bio-measurement data recording device to be able to measure people of different heights, there are preferably so many back surfaces available that for every height between, for example, 1.5 and 2 m, it is always possible to select back surface sensors by means of which a standard lead is feasible. In particular, eight back surface sensors are available.

The back surface sensors and/or the seat surface sensors preferably refer to capacitive sensors. These have proven to be especially well-suited for determining an ECG.

The sensors, including the hand grip sensors, are arranged such that, along with the back surface sensors and the seat surface sensors, seven standard leads are available for the electrocardiogram. It has been proven that these seven leads are normally sufficient for detecting any heart diseases. If an anomaly occurs upon the evaluation of the seven leads, a warning can be issued, according to a preferred embodiment. A complete electrocardiogram can then be conducted on the person using a conventional exercise ECG device.

According to a preferred embodiment, there are so many sensors available and they are arranged in such a way that at least a 7-channel-ECG can be ascertained. In particular, there are so many sensors available and they are arranged in such a way that at least seven leads can be taken from the person for the creation of the ECG. ECGs with less channels do not generally deliver measurement data that has a sufficient diagnostic value. It is still possible to detect changes in heart rate, for example, and/or record serious disruptions in cardiac activity, but further diagnostic statements are almost impossible.

According to a preferred embodiment, the bio-measurement data recording device comprises an ergometer unit. For example, the ergometer unit may be a pedal ergometer unit which can be locked in different positions relative to the seat. In this way, the ergometer unit can be adjusted to the length of the person's leg. The person can sit in the seat, independent of their height. This ensures that an ECG can always be recorded.

An ergometer unit should be understood to mean a device by means of which a physical output produced by the person can be measured. In particular, the ergometer unit is connected to the evaluation unit, the evaluation unit being configured to automatically record whether the ergometer unit is activated and/or which outputs produced by the person or the ergometer unit are measured.

The bio-measurement data recording device preferably has a display unit which comprises a projection unit with a projection surface, especially a curved projection surface, and a projector that is designed to project images onto the projection surface. It is especially advantageous if the projection surface is at least essentially spherically curved, particularly hemispherically. The projector is configured such that that image of the person projected into the projection surface is perceived to be undistorted. It is possible, but not necessary, that the projection unit is configured to project three dimensional images.

The projection surface is preferably arranged on a projection body. The projection body preferably has a concave inner contour, for example curved on two levels, which preferably follows the surface of a sphere, at least in essence and/or at least in sections. This facilitates the projection of the image. However, it is also possible to use a projection body with a different inner contour, such as an elliptical inner contour.

According to a preferred embodiment, the projection unit has a projection body that comprises at least three segments that are connected to one another. The projection body preferably comprises a plurality of segments, especially at least 3, preferably at least 7 segments.

It is especially beneficial if the segments are designed to lock positively with each other. In this way, the segments have at least one protruding structure which engages with at least one retreating structure of the segment that is arranged next to it. This creates a particularly stable projection body.

The segments may be made of dimensionally stable foam material, for example foamed polystyrene. The segments are preferably designed such that surfaces located next to each other are positively connected, or fit closely with respective lateral surfaces such that they support each other. In order to disturb the projection as little as possible, the inward-facing concave side of the projection body preferably has a film that lies on the segments.

It is preferable if an extensive elastic material, especially a textile, covers and/or stretches over the segments, at least on the convex side. The textile may refer to an elastic fabric or an elastic mesh, which is often more beneficial. It is particularly favourable if the segments are too stretched over each other through the textile. It is then possible to connect the segments to each other using the textile, which makes them easier to disassemble and assemble, thus rendering transport easier. In other words, the projection unit has at least one elastic textile clamping element, which is at least partially arranged around the segments such that these are fixed to one another. For example, the clamping element comprises a Velcro fastener by means of which it can be fixed on or relative to the elements.

According to a preferred embodiment, the projection unit comprises a film whose contour corresponds to an inner contour of the projection body and which is fixed radially inside the projection body. The film preferably undergoes a finishing treatment to increase the surface tension such that it has a surface tension of at least 37 mN/m (milli—Newton per meter). This can be achieved using, for example, a flame treatment and/or corona treatment. For example, the film is produced by deep-drawing. The film is preferably made of an elastomer, especially a thermoplastic. In order to obtain particularly good projection properties, it is advantageous if the film is white.

In order to increase the stability of the projection unit, the projection body preferably comprises a frame, for example made predominantly of metal, to which the segments are directly or indirectly fixed.

The advantage of the display unit is that the person can be shown a film that motivates them to use the equipment in a predetermined manner. For example, the display unit is configured to show a film that is designed to give the person the feeling that they are cycling through the countryside. The recording of the electrocardiogram is then no longer perceived as a medical activity, but rather as something fun.

The projection surface is preferably curved such that the person is sitting at least predominantly inside an imagined sphere that fits to the projection surface. This reduces the person's susceptibility to external influences.

It is especially advantageous if the ergometer unit is mechanically decoupled from the projection unit. This should be particularly understood to mean that both units are coupled with one another such that a vibration of the ergometer unit, which occurs during its use, is so greatly reduced that the naked eye cannot detect any vibration of the projected image.

For example, the display unit can be moved relative to the seat and/or the ergometer unit. This means that it can be planned for the display unit on the one side and/or the seat or the ergometer unit on the other to be situated on the ground or on top of each other. By placing the bio-measurement data recording device on a sufficiently rigid base, for example on a concrete floor, the decoupling between the display unit, especially the projection unit, on the one side and the ergometer unit on the other can be achieved.

According to a preferred embodiment, the bio-measurement data recording device comprises at least one, especially two, three, four or a plurality of bio-measurement data recording devices, which are selected from the group that comprises: a blood measurement device, a heart rate measuring device, a body fat sensor, a bone density measurement device, a spirometer, a blood oxygen measurement device, a hearing test device, a vein capacity measurement device and a weight determination device for determining the person's weight. In particular, the evaluation device is such designed that at least a plurality of the named devices can be controlled by the evaluation device and the delivered measurements can be read. The bio-measurement data recording device can then be configured in this way and adjusted to the respective requirements.

The evaluation unit preferably comprises a master programme, i.e. a superior programme, and independent sub-modules, which are called up by the master programme. The above named sub-modules operate the above named measurement devices, for example the blood measurement device of the heart rate measurement device, and deliver the measurement data that they have recorded. For example, the sub-modules run in the individual measurement devices, which are connected to the evaluation unit via an interface, such as a cable. As a result of this hierarchical structure, only a defined data record will be transferred to the sub-module. This ensures that an approval of the sub-module by a health authority shall not be influenced by the control by means of the master unit.

According to a preferred embodiment, the left hand grip and/or the right hand grip has at least one activation element and the evaluation unit is connected to the at least one activation element and designed for the automatic execution of a method with the steps: (i) control of the display unit such that the display unit shows a selection menu, and (ii) recording of a selection from the selection menu by recording an activation of at least one of the activation elements, (iii) the activation elements being arranged for activating the at least one activation elements during uninterrupted contact with the corresponding hand grip sensor. In other words, the activation element can be activated without losing contact between the hand and the hand grip sensor. This has the advantage that the ECG can be permanently recorded, even when the person is operating the activation element.

The evaluation unit is preferably configured to automatically carry out a method with the steps: recording of the leads from the sensors, creation of an electrocardiogram based on the leads, and, where appropriate, ascertainment of whether the electrocardiogram indicates a health risk and provision of a warning signal to the person, if a health risk is ascertained.

According to a preferred embodiment, the evaluation unit is configured to automatically carry out a method with the steps (i) recording of a sequence of bio-measurement data recording orders by repeatedly recording a selection from the selection menu and (ii) for every bio-measurement data recording order, control of the bio-measurement data recording device that is assigned to the bio-measurement data recording order such that the bio-measurement data recording orders are successively processed, and for every bio-measurement data recording order, saving of the recorded bio-measurement data. In other words, an individual check-up can be programmed via the user's selection.

Alternatively or additionally to this, it is possible that the sequence of bio-measurement data recording orders is predetermined. For example, the bio-measurement data recording device has a chip card reader and the sequence is saved on a chip card, which the user inserts into the chip card reader so as to identify themselves. It is possible for the recorded bio-measurement data to be saved on the chip card in an encrypted form.

The display unit is preferably configured to project a film onto the hemispherical screen. The user then has the feeling that they are in the projected scene. Their thoughts are now within the scene. Data such as time, length and performance data can be superimposed onto the projection surface. The prevention ergometer and its applications are then preferably designed to be controlled via the touchscreen.

In the following, the invention will be explained in more detail with the aid of drawings. They show

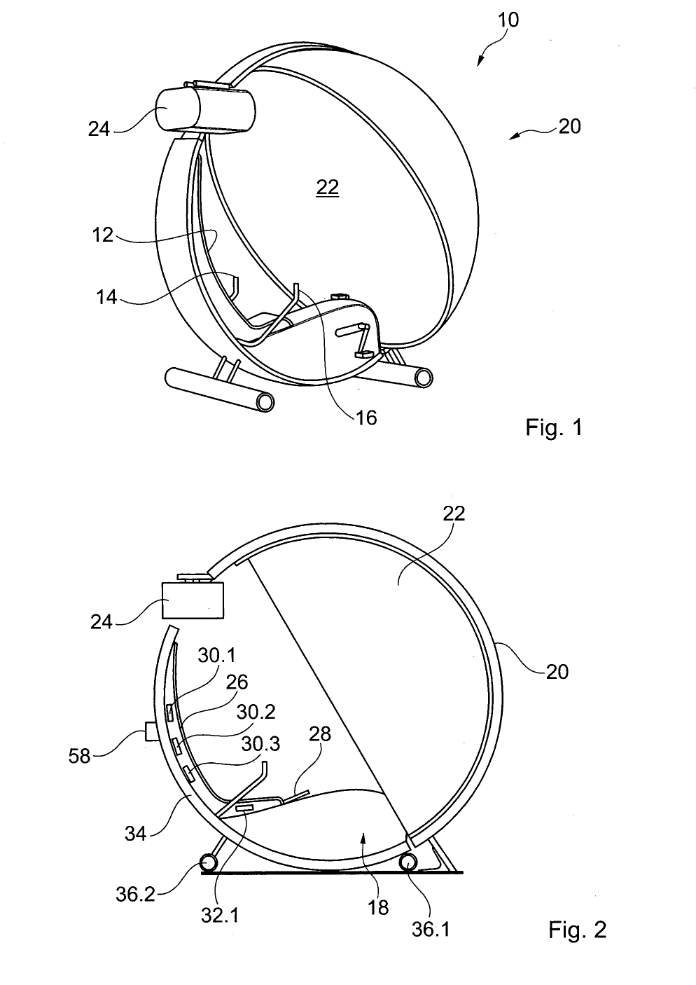

FIG. 1 a bio-measurement data recording device according to the invention in a three dimensional view,

FIG. 2 a side view of the bio-measurement data recording device according to FIG. 1,

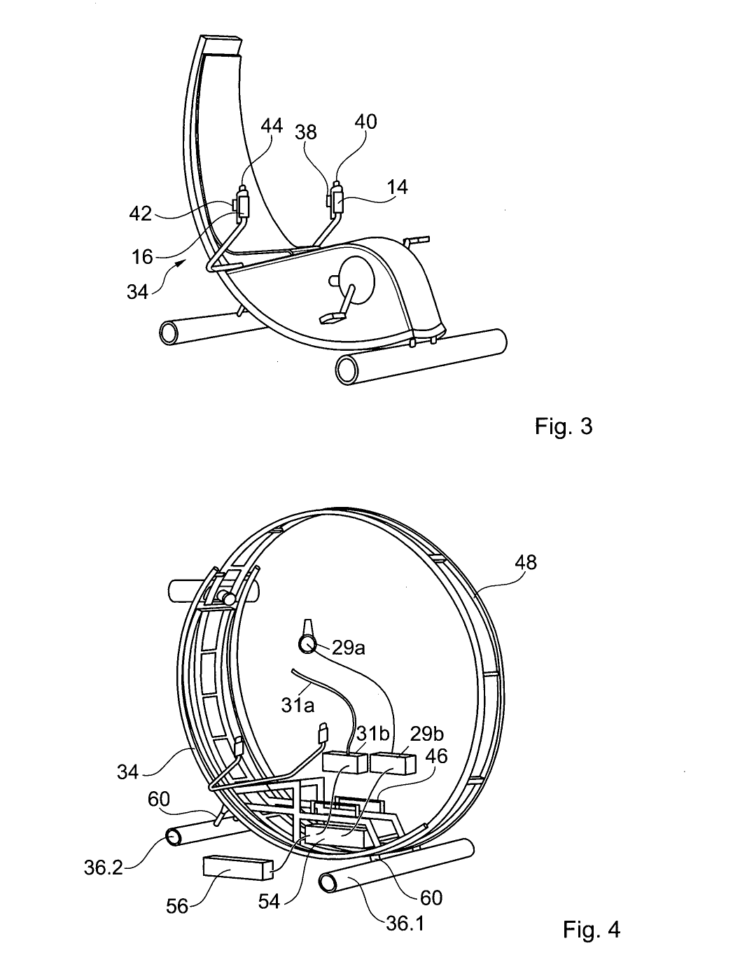

FIG. 3 a detailed view of the seat and the ergometer unit,

FIG. 4 the basic structure of the bio-measurement data recording device,

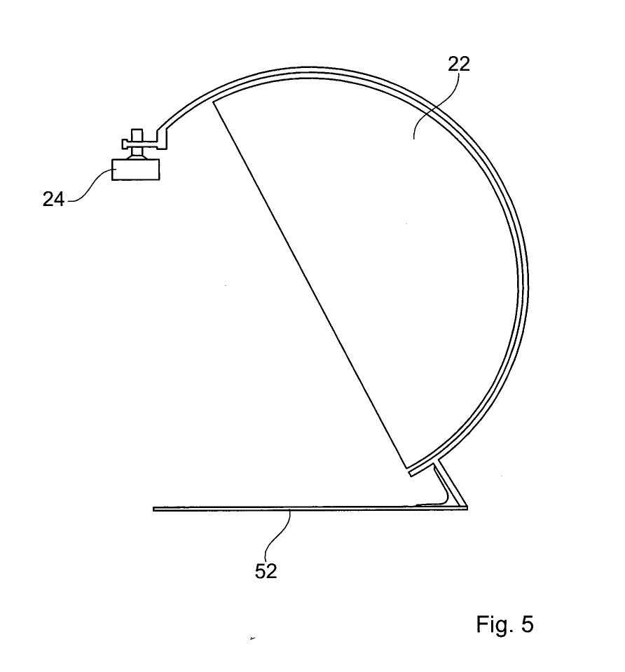

FIG. 5 a view of the display unit and

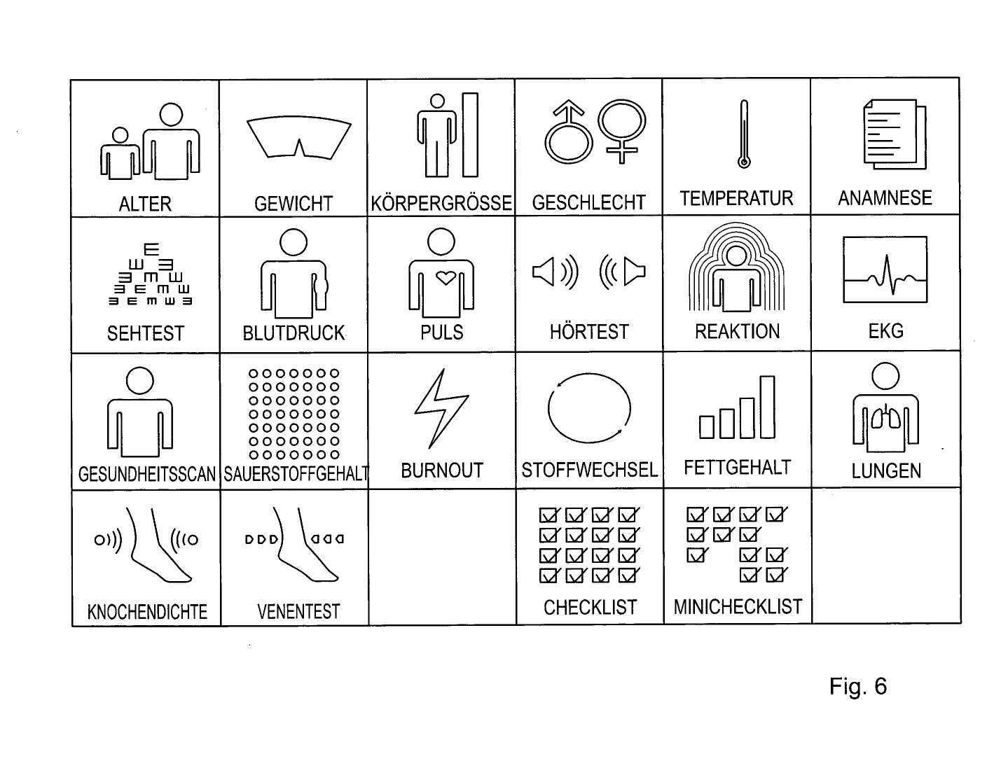

FIG. 6 an example of a selection menu which is shown by the evaluation unit during operation.

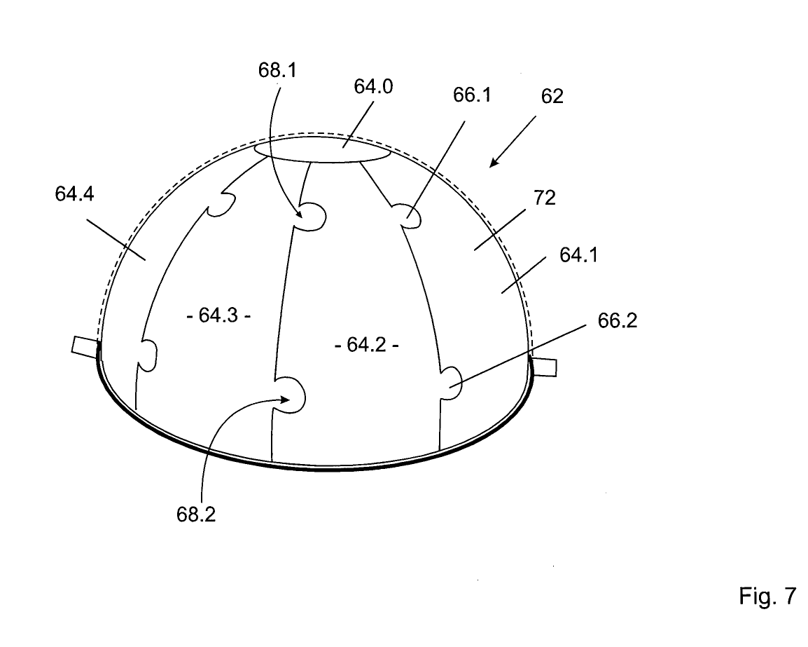

FIG. 7 shows a schematic view of the structure of a projection body made of several segments.

FIG. 1 depicts a bio-measurement data recording device 10 according to the invention, which comprises a seat 12, a left hand grip 14, a right hand grip 16 and an evaluation unit, not depicted in FIG. 1. The bio-measurement data recording device 10 also has an ergometer unit 18 in the form of a pedal ergometer unit that is arranged such that a person sitting on the seat 12 can activate it.

The bio-measurement data recording device 10 also has a display unit 20 which, alongside a touchscreen, not depicted in FIG. 1, comprises a projection unit with a projection surface 22 and a projector 24. In the present case, the projection surface 22 is designed as a hemisphere, and the projector 24 projects an image which is distorted corresponding to the curve of the surface, such that the person sitting on the seat 12 sees an undistorted image.

FIG. 2 shows a cross-section through the bio-measurement data recording device 10. It should be noted that the seat comprises a back surface 26 and a seat surface 28. The back surface and seat surface are covered by a viscoelastic foam material into which sensors are embedded—these sensors are schematically depicted. The back surface sensors 30.1, 30.2, . . . , 30.6 are embedded in the back surface 26 in this way, the back surface sensors 30.4, 30.5, 30.6 being concealed by the back surface sensors 30.1, 30.2 and 30.3. The back surface sensors 30 (reference numbers with no numerical suffix refer to all such objects) are connected to the display unit, not depicted in FIG. 2.

Seat sensors 32 are arranged in the seat surface 28, but only the seat surface sensor 32.1 is schematically depicted. A total of, for example, four seat surface sensors 32 are provided, which are all connected to the non-depicted display unit, for example by cable.

The pedal ergometer unit 18 is fixed to a frame 34 onto which the seat 12 is also arranged. The frame 34 stands on the ground by means of two feet 36.1, 36.2.

FIG. 3 shows a perspective view of the frame 34. It should be noted that the left hand grip 14 has a schematically drawn hand grip sensor 38 and a left activation element 40 in the form of a button. The activation element 40 can be activated using the thumbs without breaking contact with the hand grip sensor 38. Similarly, the right hand grip 16 has a right hand grip sensor 42 and a right activation element 44. Along with the back surface sensors, not depicted in FIG. 3, and the seat sensors, a 7 channel electrocardiogram can be accommodated, which is accommodated by the display unit, not depicted in FIG. 3.

FIG. 4 shows the frame 34 without the seat. It should be noted that the frame has a holder 46 which is arranged on the ergometer unit 18 (cf. FIG. 1) such that can be moved and fixed in position. The frame 34 has a closed frame 48 which is designed in the shape of an arc in the present case and enables an especially advantageous flux.

FIG. 5 shows a projection unit 50 which is part of the display unit and comprises the projector 24 and the projection surface 22. The projection unit 50 has a foot 52 that is designed such that the foot 36 (cf. FIG. 4) does not touch the foot 52, thereby achieving a mechanical decoupling. It is particularly beneficial if either the foot 36 or the foot 52 comprises a damping element, for example a foam material casing, so that vibrations from the ergometer unit 18 are not transmitted to the projector 24.

FIG. 4 schematically depicts the evaluation unit 54 which is connected to the sensors 30, 32, 38, 42, the activation elements 40, 44 and the projector 24. The evaluation unit 54 is connected to at least one bio-measurement data recording device 56, not depicted in FIG. 4, via a non-depicted electrical connection. This bio-measurement data recording device 56 may refer to a bone density measurement device, for example.

In addition to this, one should note schematically depicted headphones 29a of a hearing test device 29b, which is connected to the evaluation unit 54, and a spirometer 31b with a breathing tube 31a, which is also connected to the evaluation unit 54.

The mode of operation for the bio-measurement data recording device is described in the following. After starting the device, for example by inserting the chip card into a chip card reader 58 (cf. FIG. 2), the evaluation unit 54 controls the projector 24 such that, for example, the menu depicted in FIG. 6 is projected onto the projection surface 22. The user can select individual buttons using the activation elements 40, 44, thereby starting the programme. For example, the age is requested first of all. The bio-measurement data recording device 10 may also have a voice output device, for example one of the speakers, not depicted in the figure.

An instruction for how to enter the age is given via this speaker. A weighing device 60, schematically depicted in FIG. 4 and arranged in the feet 36.1, 36.2, is used to record the person's weight. As the person has to hold the activation elements in their hand to use the menu, an electrocardiogram (ECG) can be created via the sensors 38, 42, 30 and 32 of the evaluation unit 54. Depending on the programme sequence, an automatic demand may also be given to activate the ergometer unit 18. The evaluation unit 54 controls the projector 24 such that a film is shown which gives the person the impression of riding a real bike.

Following this, the person may select, for example, a bone density measurement. The evaluation unit 54 then provides instructions, by means of the projector 24, of how this measurement is taken

It is particularly favourable if the evaluation unit 54 is configured to record which bio-measurement data recording devices are connected. This may occur by requesting device identifications via an electronic bus.

A blood pressure cuff is not depicted, by means of which the blood pressure can be recorded. The blood pressure cuff may also include a body fat sensor for measuring the fat content of the body.

In the field of medicine, a contactless exercise ECG is conducted with a lung test, for example, on the prevention ergometer, wherein capacitive sensors are located in the back section of the integrated seat in the posterior area and on the integrated hand grips. In medical use, a hearing test is conducted via separate headphones and, following a further request, a sight test is projected on the hemispherical screen. The bone density and the vein capacity can be called up on the prevention ergometer in the static state via a separate application.

The automatic prevention ergometer according to the invention with an integrated hemispherical projection is used to individually measure all necessary body data, such as weight, body mass index (BMI), height, heart rate and blood pressure, bone density, visual acuity and hearing. A theoretical vitality value can be transmitted via the evaluation unit. The prevention ergometer programme is then individually set to the user's physical condition. Four different programmes can be selected here: the medical programme, the fitness programme, the competitive sport programme or the weight loss programme. During the programme sequence, the prevention ergometer is regulated exclusively by the heart rate. If the heart rate goes over the threshold value, the force on the pedals is automatically reduced until the heart rate is once again at a safe level. To this end, it is especially important that the two heart rate sensors that are arranged in the upper palm area of the ergonomically shaped hand grips simultaneously function as two of the in total seven sensors for the 5 channel exercise ECG. The heart rate is permanently measures and recorded.

FIG. 7 schematically depicts the structure of a projection body 62 made up of several segments 64, of which the segments 64.1, 64.2, 64.3, 64.4 and 64.0 are visible. Two further segments are concealed and thus not visible. In total, the projection body 62 comprises seven segments. However, it is also possible that the projection body 62 has more than seven segments, for example nine, eleven or more. For example, it is also possible that the projection body 62 has an even number of segments. All of the segments 64 (reference numbers without a numerical suffix refer to the corresponding object as a whole) but one are structurally identical, thus rendering them easy to produce. It should be noted that all the segments are positively connected to one another. In addition to this, every segment, for example the segment 64.2, has at least one protrusion 66, in the present case the two protrusions 66.1 and 66.2, as well as at least one recess, in the present case the recesses 68.1 and 68.2. Each protrusion engages with a recess 68 so that the projection body 62 is particularly structurally stable. It is of course possible to connect the segments to one another in a different way, for example using separate connection elements. The segment 64.0 is arranged such that it is in contact with all other segments and is situated in a polar region of the projection body 62 which, in the present case, is designed in the shape of a hemisphere.

It should be noted that the segments 64 are connected to a frame 70, such that the projection body 62 can be moved by means of the frame 70.

A textile clamping element 72 is schematically depicted which, in the present case, comprises a stretch material. The clamping element is placed around the segments 64 from the outside and presses them together in the circumferential direction. It is thereby possible to keep the segments connected to one another using only the clamping element. It is of course possible to provide further connection elements and/or clamping element, in addition to the clamping element, or to forego the clamping element completely. However, it has been proven that a projection body with a clamping element 72 is especially simple to produce and transport.

A film which forms the projection surface 22 (cf. FIG. 1) is not visible in FIG. 7. This film is produced, for example, by deep-drawing and is made of an elastomer, in particular a thermoplastic. The film is designed such that it sticks to the segments 64. For example, the segments 64 are made of a foamed plastic, especially foamed polystyrene. In particular, the film is surface treated such that it sticks to segments from the inside. This type of finishing treatment may be a corona treatment, for example. The finishing treatment is conducted such that a surface tension is preferably greater than 38 million Newton-metres. In this case, the film sticks to the segments. Of course it is possible, alternatively or additionally, to firmly connect the film to the projection body 62, for example using adhesive.

Reference list

| | |
|---|---|
| 10 | Bio-measurement data recording device |
| 12 | Seat |
| 14 | Left hand grip |
| 16 | Right hand grip |
| 20 | Display unit |
| 22 | Projection surface |
| 24 | Projector |
| 26 | Back surface |
| 28 | Seat surface |
| 29b | Hearing test device |
| 30 | Back surface sensor |
| 31 | Spirometer |
| 32 | Seat surface sensor |
| 34 | Frame |
| 36 | Foot |
| 38 | Left hand grip sensor |
| 40 | Left activation element |
| 42 | Right hand grip sensor |
| 44 | Right activation element |
| 46 | Holder |
| 48 | Frame |
| 50 | Projection unit |
| 52 | Foot |
| 54 | Display unit |
| 56 | Bio-measurement data recording device |
| 58 | Card reader |
| 60 | Weighing device |
| 62 | Projection body |
| 64 | Segment |
| 66 | Protrusion |
| 68 | Recess |
| 70 | Frame |
| 72 | Clamping element |

1. A bio-measurement data recording device having
(a) a seat for a person, which has
a back surface made of a foam material,
a seat surface,
a plurality of back surface sensors
which are embedded in the back surface such that a plurality of leads for an electrocardiogram of the person can be received, and
at least two seat surface sensors,
which are embedded in the seat surface such that leads for the electrocardiogram of the person can be received,
(b) a left hand grip,
which is arranged such that it can be gripped by the person with the left hand, and
has a left hand grip sensor for conducting a channel for the electrocardiogram,
(c) a right hand grip,
which is arranged such that it can be gripped by said person with the right hand and
has a right hand grip sensor for conducting a channel for the electrocardiogram, and
(d) an evaluation unit which
is connected to the sensors and
is configured to ascertain the electrocardiogram.

2. The bio-measurement data recording device according to claim 1, wherein the back surface sensors and the seat surface sensors are capacitive sensors.

3. The bio-measurement data recording device according to claim 1, further comprising an ergometer unit which can be locked in different positions relative to the seat.

4. The bio-measurement data recording device according to claim 1 further comprising a display unit which comprises a projection unit having
projection surface and a projector that is designed to project images onto the projection surface.

5. The bio-measurement data recording device according to claim 3 further comprising a projection unit, and wherein the seat and the ergometer unit are mechanically decoupled from the projection unit.

6. The bio-measurement data recording device according claim 1 further comprising at least one bio-measurement data recording device selected from the group consisting of a blood measurement device, a heart rate measuring device, a body fat sensor, a bone density measurement device, a spirometer, a blood oxygen measurement device, a hearing test device, a vein capacity measurement device, and a weight determination device for determining the person's weight.

7. The bio-measurement data recording device according claim 1 wherein the evaluation unit is connected to all bio-measurement data recording devices for recording measurement data.

8. The bio-measurement data recording device according to claim 1, wherein
(a) the left hand grip and/or the right hand grip has at least one activation element, and
(b) the evaluation unit is connected to the at least one activation element and designed for the automatic execution of a method with the steps:
(i) control of the display unit such that the display unit shows a selection menu, and
(ii) recording of a selection from the selection menu by recording an activation of at least one of the activation elements,
(c) the activation elements being arranged for activating the at least one activation elements during uninterrupted contact with the corresponding hand grip sensor.

9. The bio-measurement data recording device according to claim 1 wherein the evaluation unit is configured to automatically carry out a method with the steps:
recording of a sequence of bio-measurement data recording orders by repeatedly recording a selection from the selection menu, and
for every bio-measurement data recording order, control of the bio-measurement data recording device (56) that is assigned to the bio-measurement data recording order such that the bio-measurement data recording orders are successively processed, and
for every bio-measurement data recording order, saving of the recorded bio-measurement data.

10. The bio-measurement data recording device according to claim 1 wherein the evaluation unit is configured to automatically carry out a method with the steps:
recording of the leads from the sensors,
creation of an electrocardiogram,
ascertainment of whether the electrocardiogram indicates a health risk and
provision of a warning signal to the person, if a health risk is ascertained.

11. The bio-measurement data recording device according to claim 4 wherein the projection unit has a projection body which comprises at least three segments that are connected to one another.

12. The bio-measurement data recording device according claim 4 wherein the projection unit has one textile clamping element, which is at least partially arranged around the segments such that these are fixed to one another.

13. The bio-measurement data recording device according to claim 11 wherein the projection unit comprises a film whose contour corresponds to an inner contour of the projection body and which is fixed radially inside the projection body.

14. The bio-measurement data recording device according to claim 12 wherein the projection unit comprises a film whose contour corresponds to an inner contour of the projection body and which is fixed radially inside the projection body.

15. The bio-measurement data recording device according to claim 3 wherein the ergometer unit is a pedal ergometer unit.

16. The bio-measurement data recording device according to claim 5 wherein the projection surface is curved.
